(19) United States
(12) Patent Application Publication
(10) Pub. No.: US 2021/0205118 A1
(43) Pub. Date: Jul. 8, 2021

(54) MANDIBULAR REPOSITIONING DEVICE

(71) Applicant: Christopher Kelly, Sydney, NSW (AU)

(72) Inventor: Christopher Kelly, Sydney, NSW (AU)

(21) Appl. No.: 16/087,351

(22) PCT Filed: Apr. 5, 2017
(86) PCT No.: PCT/AU2017/050287

§ 371 Date: Sep. 21, 2018

(30) Foreign Application Priority Data
Apr. 7, 2016 (AU) 2016901292

(51) Int. Cl. A61F 5/56; A61C 7/36

(52) U.S. Cl. CPC A61F 5/566; A61C 7/36

(57) ABSTRACT

Described herein is a mandibular repositioning device (MRD). The MRD includes an upper dental plate shaped to fit on a person's maxillary arch and a lower dental plate shaped to fit on the person's mandibular arch, the plates defining mating bite surfaces. The MRD includes a locating mechanism for effecting antero-posterior (AP) correction of the mandibular arch relative to the maxillary arch. The locating mechanism includes a locating formation extending from one of the upper dental plate and the lower dental plate, and a complementary receiving formation carried by the other of the upper dental plate and the lower dental plate. The receiving formation receives the locating formation for locating the plates relative to each other in a desired AP position of the mandibular arch relative to the maxillary arch. The MRD also includes a positioning element received at each molar region of at least one of the plates to alter the bite surface, thereby effecting a natural relative movement of the dental arches. The locating mechanism is positioned to lie anterior to a user's masseter muscle complex.

### TECHNICAL FIELD

The present disclosure relates, generally, to a mandibular repositioning device and, more particularly, to a mandibular repositioning device that delimits allowable relative mandibular movement.

### BACKGROUND

A mandibular advancement device (MAD), also called a mandibular repositioning device (MRD), is a device or splint worn in the mouth to treat orofacial disorders, such as snoring, by moving the mandibular arch slightly forward. MRDs are reported to have varying efficacy based on a number of reasons typically associated with the construction of the specific device. Some problems associated with MRDs include the material used being too rigid and uncomfortable, the device being too bulky, or the device overly restricting movement or night-time swallowing.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

One problem of existing MRDs is that they may not be able to withstand lateral bruxing (teeth grinding, particularly during sleep). A user that bruxes while using an MRD may cause the material of the MRD to wear away, and this will result in the MRD not functioning properly. One way of addressing this is to consider the opposing stamp cusp-fossa relationship that determines how upper and lower teeth sit and move against one another. Existing devices generally provide opposing surfaces that do not mimic the user's natural stamp cusp relationship, especially with an altered mandibular position, resulting in discomfort and limited mobility.

Another problem with existing MRDs is that they may not enable or support comfortable swallowing while a user is asleep. In particular, swallowing is not supported at the vertical that is appropriate for the particular user. The vertical dimension is the length of the face determined by the distance of separation of jaws. Occlusal vertical dimension (OVD) or contact vertical dimension is the lower face height with the teeth in centric occlusion. Rest vertical dimension (VDR) is the lower face height measured from the chin to the nose, with the mandible in rest position. One way of addressing swallowing is to limit the intake of air into the oral cavity by providing a shield across the labial side of the MRD. Another problem with existing MRDs is that a potential first-time user is not able to trial a low-cost version of an MRD that fits the user and functions properly. A generic and adjustable MRD may be used to address this.

Another problem with existing MRDs is that the tab and slot combinations used to hold the upper and lower portions together are prone to “fin slip”. Fin slip occurs when the tab slides around within the slot, or slips out of the slot, and gets stuck in an incorrect position. This is typical where inserts are used in the dental portions of the MRDs, especially when the inserts are made of a harder or less flexible material than the base material used for the dental portions that receive the inserts. The tabs tend to slide over or around the inserts, and get stuck in an undesirable position.

In consideration of existing deficiencies in MRDs, described herein are a number of embodiments of mandibular devices that provide some improvement over existing MRDs.

In one aspect of the disclosure there is provided a mandibular repositioning device (MRD) which includes: an upper dental plate shaped to fit on a person's maxillary arch; a lower dental plate shaped to fit on the person's mandibular arch, the plates defining mating bite surfaces; a locating mechanism for effecting antero-posterior (AP) correction of the mandibular arch relative to the maxillary arch, the locating mechanism including a locating formation extending from one of the upper dental plate and the lower dental plate and a complementary receiving formation carried by the other of the upper dental plate and the lower dental plate, the receiving formation receiving the locating formation for locating the plates relative to each other in a desired AP position of the mandibular arch relative to the maxillary arch; and a positioning element received at each molar region of at least one of the plates to alter the bite surface, thereby effecting a natural relative movement of the dental arches. The locating mechanism may be positioned to lie anterior to a user's masseter muscle complex.

As used herein, the term “natural” when referring to a natural relative positioning of the mandibular and maxillary arches refers to a normal resting position of a person's jaws without a mandibular repositioning device, where the person's teeth are typically spaced slightly apart or, in other words, an “at rest” positioning of the jaws relative to each other. The normal resting position may be understood with reference to the freeway space, also generally referred to as interocclusal distance or interocclusal rest space. Freeway space is the space between the occluding surfaces of the maxillary and mandibular teeth when the mandible is in physiologic resting position, and is defined as the difference between the VDR and the OVD.

Each positioning element of at least one of the dental plates may include a bearing member shaped to effect the natural relative movement of the dental arches by guiding the movement of the person's mandible relative to the person's maxilla. The bearing member may be positioned so as to provide a pivot point for deflecting force resulting from masseter-temporalis contraction

The locating formation may comprise a pair of spaced locating tabs extending from the one of the plates and wherein the complementary receiving formation comprises a pair of spaced receiving formations carried by the other of the plates.

Each locating tab may be positioned at a molar region of its associated plate and each receiving formation is, similarly, positioned at a molar region of its associated plate.

Each receiving formation may define a slot having an AP dimension that exceeds an AP dimension of its associated locating tab to allow AP mandibular movement of the mandibular arch relative to the maxillary arch.

Each locating tab may have a length that allows relative vertical movement of the locating tab within its respective slot without the locating tab dislodging from the slot. Each of the locating tabs may extend from the one of the plates from a front of the tab to a back of the tab to form a closed tab.

Each positioning element may define an occlusal surface, the shape of the occlusal surface being based on anatomic curvature of the occlusal alignment of teeth of the person. Each positioning element of at least one of the dental plates may be selected with its occlusal surface standing proud of the bite surface of its associated dental plate by a selected amount. The predetermined distance may be selected based on the person's bite classification.

Each positioning element associated with the dental plate having the receiving formation may include an auxiliary receiving formation. The auxiliary receiving formation may be shaped to lie along substantially an entire length of its respective receiving formation.

The MRD may further include a shield shaped to be positioned relative to a labial region of the dental plates.

Each dental plate may comprise: a pair of discrete molar components, each molar component carrying one of the locating formations or one of the receiving formations, and each molar component being shaped to receive one of the positioning elements; and an adjustment mechanism that adjustably couples the pair of molar components together.

The adjustment mechanism may comprise a pair of coupling members, one carried by each molar component, the members being adjustably connectable together to vary the spacing of the molar components from each other.

In another aspect of the disclosure there is provided a mandibular repositioning device (MRD) which includes: an upper dental portion including a pair of discrete upper molar components, shaped to fit on a person's maxillary arch, and a first adjustment mechanism that adjustably couples the pair of upper molar components; a lower dental portion including a pair of discrete lower molar components, shaped to fit on a person's mandibular arch, and a second adjustment mechanism that adjustably couples the pair of lower molar components together, the components of the upper and lower dental portions in register with each other defining mating bite surfaces; a locating mechanism for effecting antero-posterior (AP) correction of the mandibular arch relative to the maxillary arch, the locating mechanism including: a pair of locating formations, one extending from each component of one of the pair of upper molar components and the pair of lower molar components, and a pair of complementary receiving formations, one carried by each component of the other of the pair of upper molar components and the pair of lower molar components, the receiving formations receiving the locating formations for locating the associated components of the portions relative to each other; and a positioning element received by each molar component to alter the bite surface, thereby effecting a natural relative movement of the dental arches. The positioning element may include a bearing member positioned so as to provide a pivot point for deflecting force resulting from masseter-temporalis contraction

The bearing member may be shaped to effect the natural relative movement of the dental arches by guiding the movement of the person's mandible relative to the person's maxilla.

In another aspect of the disclosure there is provided a mandibular repositioning device (MRD) which includes: an upper dental plate shaped to fit on a person's maxillary arch; a lower dental plate shaped to fit on the person's mandibular arch, the plates defining mating bite surfaces; a locating mechanism for effecting antero-posterior (AP) correction of the mandibular arch relative to the maxillary arch, the locating mechanism including a locating formation extending from one of the upper dental plate and lower dental plate and a complementary receiving formation carried by the other of the upper dental plate and the lower dental plate, the receiving formation receiving the locating formation for ideating the plates relative to each other; a positioning element received at each molar region of at least one of the plates to alter the bite surface, thereby effecting a natural relative movement of the dental arches; and a shield shaped to be positioned relative to a labial region of the dental plates. The positioning element may include a bearing member positioned so as to provide a pivot point for deflecting force resulting from masseter-temporalis contraction.

The locating mechanism and the positioning elements may be configured to allow mandibular movement. One or more of the positioning elements may be removable.

Each positioning element associated with the dental plate having the locating formation may include an auxiliary locating formation.

The locating formation may include a sleeve configured to adjust a length of the location formation.

In another aspect there is provided a mandibular repositioning device (MRD) which includes: an upper dental plate shaped to fit on a person's maxillary arch; a lower dental plate shaped to fit on the person's mandibular arch, the plates defining mating bite surfaces; a locating mechanism for effecting antero-posterior (AP) correction of the mandibular arch relative to the maxillary arch, the locating mechanism including a locating formation extending from one of the upper dental plate and the lower dental plate and a complementary receiving formation carried by the other of the upper dental plate and the lower dental plate, the receiving formation receiving the locating formation for locating the plates relative to each other in a desired AP position of the mandibular arch relative to the maxillary arch; and a positioning element received at each molar region of at least one of the plates to alter the bite surface, thereby effecting a natural relative movement of the dental arches, wherein each positioning element of at least one of the dental plates includes a bearing member positioned so as to provide a pivot point for deflecting force resulting from masseter-temporalis contraction.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the disclosure are now described by way of example with reference to the accompanying drawings in which:—

FIG. 1 is a front perspective view of an embodiment of a mandibular repositioning device (MRD);

FIG. 2A is a schematic representation of Wilson and Spee curves;

FIG. 2B is a schematic representation illustrating the occlusal vertical dimension (OVD) and the rest vertical dimension (VDR)

FIG. 3A is bottom perspective view of an upper dental plate of the MRD of FIG. 1;

FIG. 3B is a top perspective view of a lower dental plate of the MRD of FIG. 1;

FIG. 4A is a perspective view of a first embodiment of a pair of upper positioning elements;

FIG. 4B is a perspective view of a second embodiment of a pair of upper positioning elements;

FIG. 4C is a perspective view of a first embodiment of a pair of lower positioning elements;

FIG. 4D is a perspective view of a second embodiment of a pair of lower positioning elements;

FIG. 5 shows a plan view of another embodiment of a MRD;

FIG. 6A is a perspective view of a first discrete upper molar component of an upper dental portion of the MRD of FIG. 5;

FIG. 6B is a perspective view of a second discrete upper molar component of the upper dental portion of the MRD of FIG. 5;

FIG. 6C is a perspective view of a first discrete lower molar component of a lower dental portion of the MRD of FIG. 5;

FIG. 6D is a perspective view of a second discrete lower molar component of the lower dental portion of the MRD of FIG. 5;

FIG. 7 is a perspective view of a further embodiment of a MRD;

FIG. 8 is a side view of an external support for a MRD; and

FIG. 9 is a perspective view of another embodiment of a MRD.

In the drawings, like reference numerals designate similar parts unless otherwise specified.

### DESCRIPTION OF EMBODIMENTS

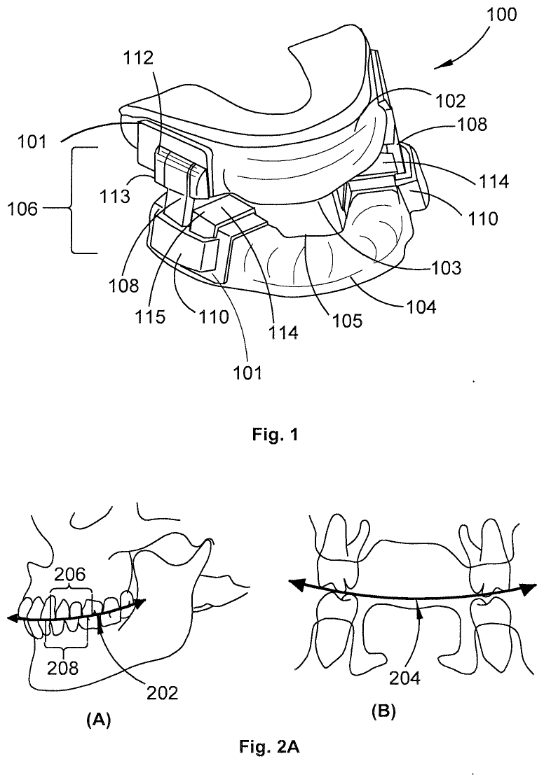

Referring to FIG. 1 of the drawings, a first embodiment of a mandibular repositioning device (MRD) 100 has an upper dental plate 102 shaped to fit on a person's maxillary arch, and a lower dental plate 104 shaped to fit on the person's mandibular arch. The dental plate 102 defines a bite surface 103. Similarly, the dental plate 104 defines a bite surface 105. The dental plates 102, 104 typically cover a biting surface of the person's teeth and are moulded using a cast of the person's teeth. The dental plates 102, 104 are held in place on the person's teeth by retention forces due to being a snug fit, and are made of a flexible permanently elastic resin (for example Ivocap™) so can easily be removed.

The MRD 100 has a locating mechanism 106 that, in this embodiment, consists of a pair of locating formations in the form of locating tabs 108, a locating tab 108 extending from a buccal side of each molar region of the upper dental plate 102. The locating mechanism 106 further includes a pair of receiving formations, a receiving formation being carried on a buccal side of each molar region of the lower dental plate 104. Each receiving formation comprises a slot-defining portion defining a slot 110. Each locating tab 108 is received in one of the slots 110.

In this embodiment, the slot-defining portion extends from a front region of a molar member 101 positioned at the bite surface in the molar region, and extends to a back region of the molar member 101. In the illustrated embodiment, the slot-defining portion is attached at both the front and back regions of the molar member 101, thereby defining a “closed” slot that does not provide a passage, e.g. towards the back of the MRD 100. Because the slot 110 is closed, fin slip is generally avoided as the tab 108 cannot easily slip out of the slot 110 through antero-posterior (AP) movement alone.

In an embodiment, the locating mechanism 106 is positioned anterior to a user's masseter muscle complex in order to effect a relative fulcrum in relation to the temporo-mandibular joint complex to accommodate bruxing.

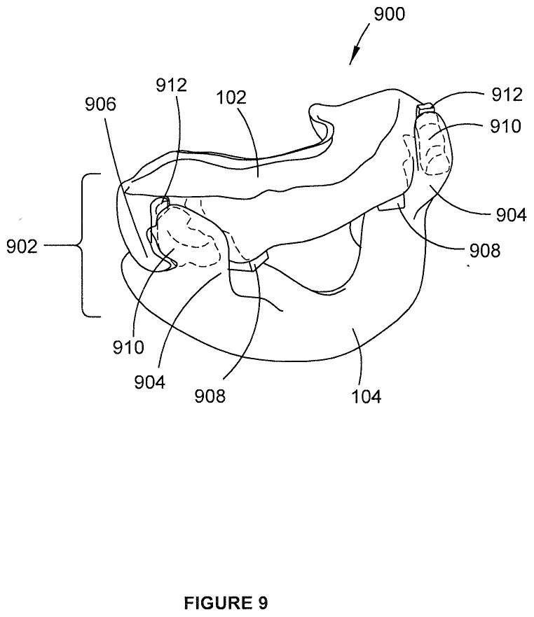

In the illustrated embodiment, the locating mechanism 106 has the tabs 108 on the upper dental plate 102 and the slots 110 on the lower dental plate 104. However, these can be swapped around (see for example the embodiment shown in FIG. 9). In some embodiments, a single tab and slot combination is used instead of a pair. In the illustrated embodiment the locating mechanism 106 is shown on the buccal side (outside) of the dental plates, however the locating mechanism may also be positioned on the lingual side (inside), although this may reduce intra-oral volume required for the tongue.

A positioning element is provided on the bite surface around the molar region of the upper and/or lower dental plates. As shown in FIG. 1 a positioning element, in the form of a removable positioning saddle 112, is removably received on or over at least the bite surface 103 of the upper dental plate 102 adjacent each tab 108. In some embodiments, a complementary positioning element, in the form of a removable positioning saddle 114, is removably received over at least the bite surface 105 of the lower dental plate 104 adjacent each slot 110. Each saddle 112, 114 defines an occlusal surface 113, 115, respectively.

In the illustrated embodiment, the saddles 112, 114 are removable, and are removably received over the respective bite surfaces 103, 105. However, it will be appreciated that, in other embodiments, the saddles 112, 114 may be permanently fixed to their associated dental plates 102, 104, respectively, or, instead, fabricated integrally with their associated dental plates 102, 104 as a one-piece unit. In this embodiment, the saddles 112, 114 are received over the respective bite surfaces 103, 105 via a molar member 101, each molar member 101 carrying a tab 108 or slot 110, and adapted to receive the respective saddle. In some embodiments, the molar members 101 may be made from a firmer material than the elastic resin of the dental plates 102, 104, for example nylon. In another embodiment, the molar regions of both the upper and lower dental plates 102, 104 themselves are shaped to include seats (not shown) for receiving the saddles 112, 114.

In some embodiments the positioning element is not received over the bite surface like a saddle, but is provided on a part of the bite surface, for example integrally formed with the bite surface of one or both dental plates, or as inserts fitted into the dental plate(s) and overlying, or being flush with, the bite surface. See, for example, the embodiment shown in FIG. 9 and described in more detail elsewhere herein.

As used herein the term “molar region” refers generally to the molar and pre-molar region. The positioning elements, for example, may be provided as appropriate according to the specific person's mouth anatomy along the molar and/or pre-molar regions.

The saddles 112, 114 are provided to support the position of the maxillary and mandibular arches when the person's mouth is closed, and may also be used to adjust a spacing between the dental arches. In use, the occlusal surfaces 113, 115 of the saddles 112, 114 bear against one another, keeping the person's dental arches spaced apart slightly. This position of the dental arches closely resembles the natural, normal resting position of a person's dental arches without using a MRD. One difference is a slightly more forward position of the mandibular arch as required to inhibit obstruction of the airway during sleep. This forward position is effected by the antero-posterior (AP) correction resulting from the shape of the dental plates 102, 104 with their respective saddles 112, 114 generally, but in particular resulting from the specific fit of the locating mechanism 106. Thus, the positioning saddles 112, 114 are specifically selected for each person's mandibular anatomy with the occlusal surface 113, 115 either flush with, or standing proud of the bite surfaces 103, 105 of the dental plate 102, 104, respectively, by a selected amount. This spaces the upper and lower dental plates 102, 104 by the required amount to accommodate that person's natural relative mandibular positioning, and in particular to resemble the person's normal vertical positioning with a natural freeway space.

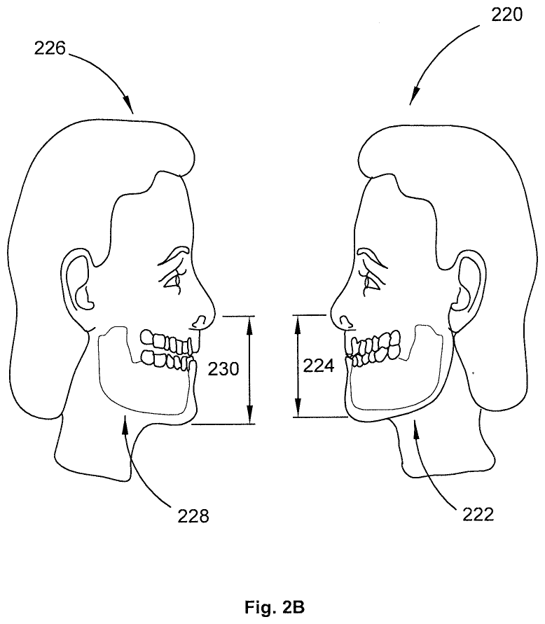

The vertical dimension is the length of the face determined by the distance of separation of jaws. Referring to FIG. 2B, the occlusal vertical dimension (OVD) 224 or contact vertical dimension is shown in the right hand FIG. 220, and is defined as the lower face height with the teeth in centric occlusion 222. The rest vertical dimension (VDR) 230 is the lower face height measured from the chin to the nose, with the mandible in rest position 228, as shown in the left hand FIG. 226. Freeway space (FWS), also generally referred to as interocclusal distance or interocclusal rest space, is the space between the occluding surfaces of the maxillary and mandibular teeth when the mandible is in a physiological resting position, and is defined as the difference between the VDR and the OVD: FWS=VDR−OVD.

The relative mandibular positioning still resembles the natural position for four reasons.

Firstly, the occlusal surfaces 113, 115 of the saddles 112, 114, respectively, are large enough so that they are maintained substantially in abutment even when the person's jaws move, whether antero-posteriorly (AP) or laterally.

Secondly, the occlusal surfaces 113, 115 are shaped so that movement of the mandible will mimic Wilson 204 and Spee 202 curves, as shown in FIG. 2A. This allows for a smooth, substantially natural action of the mandibular arch relative to the maxillary arch for lateral and protrusive bruxing movement in line with the Spee 202 and Wilson 204 curves. However full contact between the upper and lower dental plates 102, 104 is inhibited by the ribs 120. In this embodiment, having both upper saddles 112 and lower saddles 114 in opposed relationship minimises bruxing forces being disruptive to the functioning of the MRD 100 or causing structural damage to the MRD 100.

The upper saddles 112 each include a transversely extending, bearing member, in the form of a rib 120 along a front edge of the saddle, the ribs 120 configured to guide the movement of the mandible relative to the maxilla. The ribs 120 act as a pivot point positioned towards the front of the saddles 112 in order to keep any resulting forces due to, for example bruxing, away from the masseter muscle complex as activation of those muscles are to be avoided. The ribs 120 are substantially semi-cylindrically shaped, with a length substantially matching a cross-dental dimension of the respective saddle. The height and width of ribs 120 (i.e. related to the radius of the half-cylinder) are selected based on the user's specific stamp-cusp relationship of the pre-molars below the saddles. In some embodiments, ribs are provided on both the upper saddles 112 and the lower saddles 114, typically on the leading edges of both saddles. In such embodiments, the upper ribs are positioned in line with the upper first bicuspid, while the lower ribs are positioned in line with the lower second bicuspid. This positioning of the ribs results in a pivot point that not only supports natural movement of the jaws, but also avoids the transfer of force to the teeth by deflecting forces resulting from masseter-temporalis contraction. In this way the bearing members help to alleviate the effects of bruxing by reducing the resulting force experienced by the teeth.

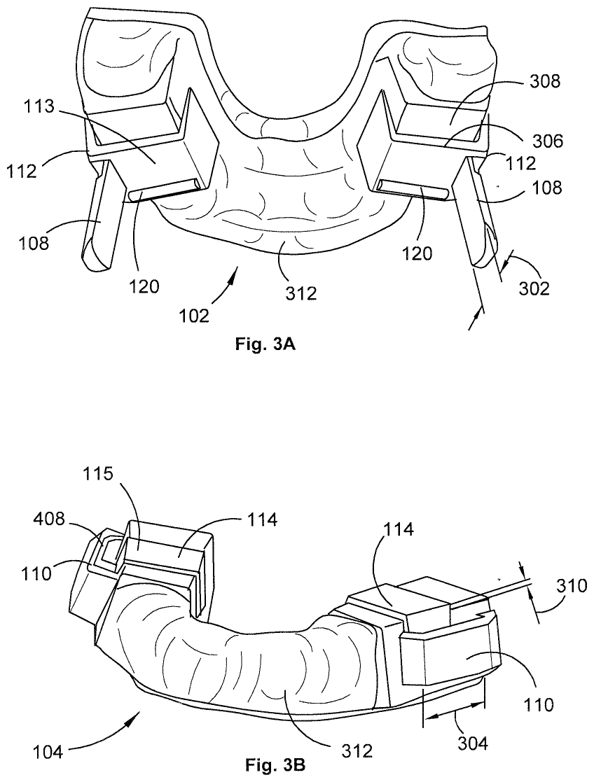

Thirdly, the slots 110 are slightly longer, in the AP direction, than the AP width of the tabs so that the mandibular arch can still move back and forth slightly (AP movement). The tab width 302 can be seen in FIG. 3A, and is typically between 5 and 15 mm, for example 9 mm. The slot length 304 can be seen in FIG. 3B, and is typically between 15 and 25 mm, for example 20 mm. The slots 110 are dimensioned to allow maximum natural physiological forward movement of the mandible. In some embodiments the slots 110 are not enclosed towards the back of the dental plate 104 (see for example the embodiment illustrated in FIG. 5).

The tab and slot arrangement provides a locating mechanism 106 that will not easily fail because some mandibular movement can be accommodated in the vertical distance that the tabs 108 can slide within the slots 110 so that the tabs 108 do not easily slide out of the slots 110. The length of the tabs 108 allow relative vertical movement of the tab 108 within their respective slots 110 without the tabs 108 dislodging from the slot 110. The two-dimensional play provided by the dimensioning of the tab and slot arrangement results in a comfortable and natural fit, allowing at least some vertical as well as AP movement.

In some embodiments the tabs 108 may include a sleeve (not shown) used to adjust the length of the tab 108 to suit the individual anatomical demands of the user and the bite registration. The sleeved tab allows for substantially free-floating contact of the slots 110 and tabs 108 while maintaining a suitable relative AP position of the lower and upper jaws. The sleeve may be made of any suitable material, for example silicon.

In some embodiments the tab and slot arrangement provides three-dimensional play. The slots 110 may be slightly wider than the thickness of the tabs, thereby allowing some lateral movement as well. This may be provided to accommodate some lateral bruxing while maintaining the tabs 108 securely within the slots 110.

Fourthly, the person's specific bite classification can be taken into consideration in order to position the maxillary and mandibular arches with the appropriate vertical alignment for the relevant classification. The bite classification may be understood with reference to the alignment shown between the upper canine and molar verticals 206 and the lower canine and molar verticals 208 in FIG. 2A. The alignment shown in FIG. 2A is a normal, class I, alignment. In a class II alignment, for example, the lower canine and molar verticals are posterior to the upper verticals, whereas in a class III alignment the lower verticals are anterior to the upper verticals. The person's bite classification can be accommodated by using an appropriate combination of positioning saddles 112, 114 with or without elevated occlusal surfaces 113, 115, as described in more detail below.

The consequences of this natural positioning and movement allowance is that on arc movement (along the curves 202, 204), postural repositioning and natural rotational advancement are all supported. In this way, both opening and forward movement of the mandibular arch are allowed, while inhibiting backwards movement to the user's habitual and anatomical resting position. This configuration of the saddles 112, 114 together with the locating mechanism 106 not only allows movement, but also delimits the movement within natural bounds and facilitates treating the relevant orofacial disorder.

In the embodiment shown in FIG. 1, FIG. 3A and FIG. 3B, both upper removable positioning saddles 112 and lower removable positioning saddles 114 are used. In other embodiments, a single pair of positioning saddles may be used, either on the upper 102 or lower 104 dental plate. In such embodiments the occlusal surfaces of the single pair of positioning saddles abuts directly against the opposing dental plate's bite surface 103, 105, as the case may be.

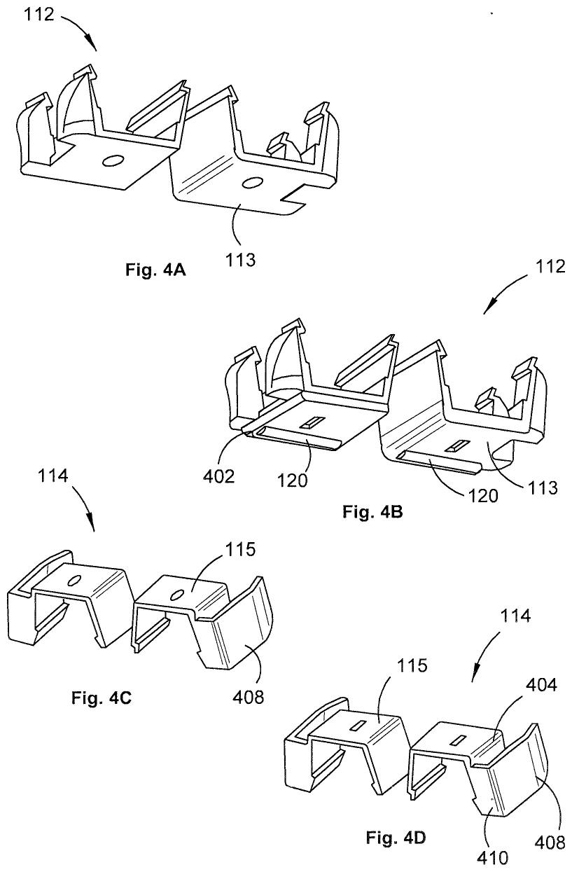

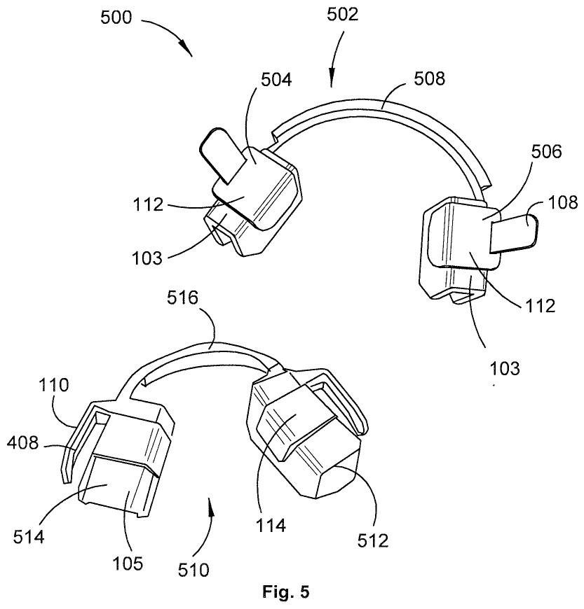

A variable spacing is effected between the dental arches through the selected thickness 310 of each pair of upper removable positioning saddles 112. Referring to the saddles 112 shown in FIG. 4A of the drawings, the upper removable positioning saddles 112 each have an occlusal surface 113 that is not, or only minimally, elevated so that, once positioned in the receiving seat of the upper dental plate 102, these “level” saddles 112 alone do not increase the spacing between the dental arches because the level saddles 112 sit substantially flush with the bite surface 103, 105 of the dental plate 102, 104. The elevated saddles 112 shown in FIG. 4B, on the other hand, each have an occlusal surface 113 on a raised platform 402, in this case raised by about 1 mm, in order to increase the spacing between the dental arches by about 1 mm.

FIG. 4C shows lower positioning saddles 114, each with an occlusal surface 115 with no, or minimal, elevation. The lower raised saddles 114 shown in FIG. 4D are similar to the ones shown in situ in FIG. 1 and FIG. 3B. These elevated positioning saddles 114 each have an occlusal surface 115 on a raised platform 404, raised by about 1 mm.

It will be appreciated that the raised platforms 402, 404 may be raised by a distance greater or less than 1 mm, depending for example on the overall construction of the MRD 100 as well as the mandibular anatomy of the person. In particular, the bite classification of the person governs the selection of saddles 112, 114 due to the specific relative mandibular positioning required for different bite classes as well as an appropriate increase or reduction in vertical dimension to avoid stress on the dental arches and the temporo-mandibular joint (which could ultimately result in arthroscopic and orthopaedic damage to the dental arches and associated structures).

For example, a class I bite may require the combination as shown in FIG. 1, FIG. 3A and FIG. 3B. A lateral bruxer with a class III posterior overjet may require a similar combination of level upper saddles 112 and elevated lower saddles 114. A person that is a clencher (as opposed to a bruxer) with a posterior class I bite would also require lower elevated saddles 112, while a clencher with a class III bite would require elevated upper saddles 112 and level lower saddles 114.

Any untreated orthodontic asymmetrical anomalies may require the use of an elevated upper saddle 112 on one side of the user's arches, and an elevated lower saddle on the other side of the user's arches. This adjustability of the MRD 100 contributes to its versatility and, hence, the efficacy of the MRD 100.

The saddles 112, 114 are typically made of a material able to withstand bruxing, for example polyamide 12 (food grade nylon), such as Grilamid L 25 W40. This material has a low hygroscopic value and thus very low water-absorption. Another example material is EOS material such as PA2200.

Sometimes it may be necessary to custom make a level saddle and/or an elevated saddle. This may be done via several methods; some suggested methods are CAD CAM production, or custom tooling within a dental laboratory. These methods are intended to be illustrative, and not restrictive. The saddles may be 3D printed using a nylon material. The upper and lower dental plates may also be manufactured with the saddles integrally formed. Such integral dental plates forming an MRD may also be 3D printed, for example using a nylon material. In some embodiments the MRD as a whole may be done using CAD production.

In some embodiments the MRD may include auxiliary locating formations (see FIG. 9, described in more detail elsewhere herein) and/or auxiliary receiving formations.

In some embodiments, as shown in FIG. 4C and FIG. 4D, each lower positioning saddle 114 includes an auxiliary receiving formation in the form of a buccal flange 408 that is shaped to fit within the slot 110 of its associated receiving formation 110. The flange 408, being made from a firmer material than the elastic resin of the dental plates 102, 104, and lying along substantially the whole length inside the slot 110, helps to keep the tab 108 in place in the slot 110. Fin slip is inhibited because the tab 110 cannot move past the flange 408 within the slot 110 as the flange 408 extends substantially along the length of the slot 110.

The flange 408 also serves to restrict the relative AP movement of the dental arches when the tab 108 is received in the slot 110, reduced in size by the flange 408, of the receiving formation. The thickness of an anterior portion 410 of the flange 408 limits AP mobility of the tab 108. Thus, the relative AP movement range of the dental arches can be set governed by the thickness of the anterior portion 410 of the auxiliary receiving formation.

In some embodiments the auxiliary receiving formation(s) and/or locating formation(s) may be provided in a separate structure not part of, or specifically associated with, the receiving and locating formations 110, 108. In one embodiment, the auxiliary receiving formation is provided in the form of a screw through or into the slot 110 of the receiving formation.

In the embodiment of a MRD 900 shown in FIG. 9, the locating mechanism 902 consists of a pair of locating formations in the form of locating tabs 904, a locating tab 904 extending from a buccal side of each molar (or pre-molar) region of the lower dental plate 104. The locating mechanism 902 further includes a pair of receiving formations, a receiving formation being carried on a buccal side of each molar (or pre-molar) region of the upper dental plate 102. Each receiving formation comprises a forward facing wing 906. Each locating tab 904 is cupped by one of the wings 906. In an embodiment, the locating mechanism 902 is positioned anterior to a user's masseter muscle complex in order to effect a relative fulcrum in relation to the temporo-mandibular join complex to accommodate bruxing.

In this embodiment the positioning element is provided in the form of a removable insert 908 received at the molar (or pre-molar) region on either side of the upper dental plate 102, altering at least a portion of the bite surface of the upper dental plate 102. The bite surfaces of each of the inserts 908 have comparable characteristics to the occlusal surface 113 of the saddles 112 in that the inserts 908 may include a raised platform to accommodate the bite classification of the user, and/or the inserts 908 may include a transversely extending bearing member, similar to rib 120, configured to guide the movement of the mandible relative to the maxilla generally along the Wilson 204 and Spee 202 curves. The inserts 908 therefore alter the bite surface in order to effect a natural relative movement of the dental arches.

Some embodiments do not have removable inserts 908 and the bite surfaces of the upper and/or lower dental plates themselves may be shaped to effect a natural relative movement of the dental arches.

In the MRD 900 AP positioning is controlled through the use of auxiliary locating formations 910 in the form of posterior inserts that fit into the posterior edge of each locating tab 904. Each auxiliary locating formation 910 includes a protruding setting portion 912 which abuts a root of its associated wing 906 to set the minimum forward position of the mandible. Where the relative AP positioning requires the mandible to be held further forward, the auxiliary locating formations 910 may be provided with a differently shaped setting portion 912.

The tab-wing configuration of the locating mechanism 902 has the added advantage of inhibiting fin slip as tabs 904 are inhibited from slipping to the outside of the wings 906, and the wing 906 is inhibited from wedging in between the tab 904 and the auxiliary locating formation 910.

A temporary or trial type embodiment of a MRD is shown in the form of a generic and adjustable MRD 500 is shown in FIG. 5.

The adjustable MRD 500 includes an upper dental portion 502 with a pair of discrete upper molar components, being a right discrete upper molar component 504 and a left discrete upper molar component 506, each shaped to fit on a person's maxillary arch. The upper dental portion 502 has an adjustment mechanism 508 (such as a pillar and hole connector) that adjustably couples the pair of upper molar components 504, 506.

The adjustable MRD 500 also has a lower dental portion 510 with a pair of discrete lower molar components, being a right discrete lower molar component 512 and a left discrete lower molar component 514, each shaped to fit on a person's mandibular arch. The lower dental portion 510 has a second adjustment mechanism 516 that adjustably couples the pair of lower molar components 512, 514 together. The adjustment mechanisms 508, 516 are adjusted so that, in use, the molar components 504, 506, 512, 514 of the upper and lower dental portions 502, 510. Respectively, are in register with each other, having mating bite surfaces.

The pillar and hole connectors 508, 516 are made from a flexible material such as silicon, and this adjustable configuration provides a simple and generic MRD that is still customisable with the saddles 112, 114 as described above with reference to FIG. 4A-4D. A positioning component such as a saddle 112, 114 is received over at least the bite surface 103, 105 of each molar component 504, 506, 508, 510. In the embodiment illustrated in FIG. 5, the saddles 114 positioned over the lower dental portion 510 include auxiliary receiving formations 408 positioned inside the slots 110. In some embodiments the saddles 112, 114 are removable, and in some embodiments one or more of the saddles 112, 114 may include an elevated platform 402, 404, respectively, as described elsewhere. The slots 110 shown in FIG. 5 are not closed towards the back of the lower dental portion 510. The slots 110 form a rearward facing wing, allowing for greater relative mobility of the dental arches as the movement of the tabs 108 are not limited when the mandible moves forward. In other embodiments, the slots 110 are closed as shown in FIG. 1 and FIG. 3B. In other embodiments the slots may form a forward facing wing, for example where the slots are provided on the upper dental plate as shown in FIG. 9 and described in more detail elsewhere herein.

The adjustable MRD 500 has a locating mechanism for effecting antero-posterior (AP) correction of the mandibular arch relative to the maxillary arch. The locating mechanism has a pair of locating formations in the form of locating tabs 108, one extending from the buccal side of each discrete upper molar component 504, 506. The locating mechanism further includes a pair of complementary receiving formations carried on a buccal side of each lower molar component 512, 514, each receiving formation comprising a slot-defining portion defining a slot 110. Each locating tab 108 is received in one of the slots 110.

The adjustment mechanisms 508, 510 are connected across the labial side of the front teeth to position the discrete molar components appropriately so that the locating mechanisms on either side are located anterior to the person's masseter muscle complex.

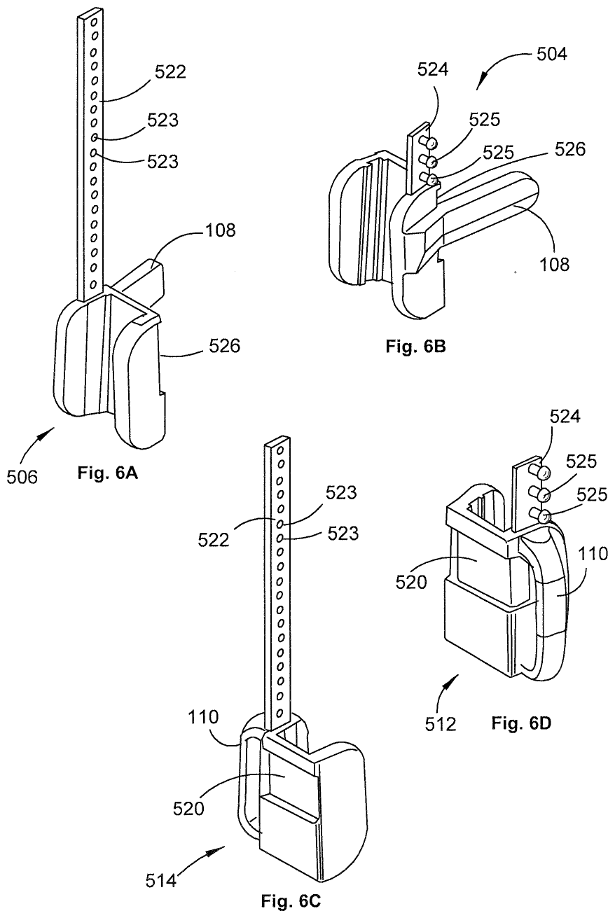

The discrete upper molar components 506, 504 are illustrated in FIGS. 6A and 6B, respectively. Each discrete upper molar component has a seat 526 positioned adjacent the locating tab 108 for receiving a saddle 112 (not shown). The adjustment mechanism 508 of the upper dental portion 502 consists of an elongate strip 522 that extends from the buccal side of the left discrete upper molar component 506. The strip 522 defines a plurality of longitudinally spaced holes 523 arranged on the longitudinal axis of the strip 522: The adjustment mechanism 508 further includes a pillar strip 524 that extends from the buccal side of the right discrete upper molar component 504. The pillar strip 524 carries a plurality of longitudinally spaced pillars 525 arranged along the longitudinal axis of the strip 524 and extending substantially perpendicularly to an outer surface of the strip 524.

The discrete lower molar components 514, 512 are illustrated in FIG. 6C and FIG. 6D. Each discrete lower molar component has a seat 520 positioned adjacent the receiving formation for receiving a saddle 114 (not shown). The adjustment mechanism 516 of the lower dental portion 510 is similar to the adjustment mechanism 508 of the upper dental portion 502 as described above. An elongate strip 522 extends from the buccal side of the left discrete lower molar component 514. The strip 522 defines a plurality of longitudinally spaced holes 523 arranged on the longitudinal axis of the strip 522. The adjustment mechanism 516 further includes a pillar strip 524 that extends from the buccal side of the right discrete lower molar component 512. The pillar strip 524 carries a plurality of longitudinally spaced pillars 525 arranged along the longitudinal axis of the strip 524 and extending substantially perpendicularly to an outer surface of the strip 524.

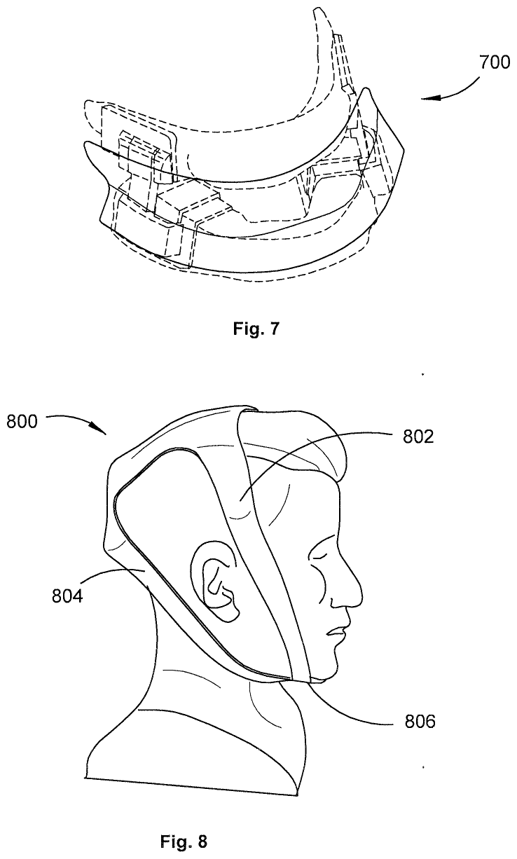

The devices described herein specifically allow mandibular movement due to the shape of the saddles and the configuration of the locating mechanism. However, to effectively treat a patient for example for snoring, it is preferably to limit mouth breathing even though the jaws can move. This can be addressed by using a shield 700 shaped to sit against a labial surface of the dental plates, as shown in FIG. 7 of the drawings. The shield 700 is made from a flexible material such as a flexible permanently elastic resin (for example Ivocap™), shaped according to the labial surface of the dental plates, sized and made according to known methods used for buccal shields generally. In this embodiment the shield 700 is unattached to the MRD 100, i.e. it is a free floating shield.

The use of the shield 700 in concert with the MRD 100 controls the extent of the movement allowed. The shield 700 is used to create a low pressure region in the oral cavity thereby causing the structures of the pharynx to be more rigid. The shield 700, when used together with the MRD 100, also helps to maintain a low pressure region in the mouth and also places the pharyngeal structures under therapeutic tension.

Being able to select appropriate combinations of saddles and saddle thickness, means that the intra-oral volume is adjustable ensuring enough space for the tongue to be in a correct position. Repositioning the tongue in concert with mandibular repositioning has beneficial effects on the pharyngeal structures by creating tension in them which in turn resists inspiratory collapse. The low pressure region brought about by the shield 700 aids in positioning the tongue. The low pressure region causes the tongue to sit in the palate with the dorsal section of the tongue against the soft palate—thus stabilizing the velum and uvula. The support provided by the low pressure region holds the dental arches in their relative positions as derived by the MRD 100, by holding the tongue against the palate and soft palate, but also allows voluntary opening of the jaw vertically within the limits of the low pressure region. The resulting seal also encourages nasal breathing.

The shield 700 makes the MRD 100 less intrusive in the user's oral cavity and more comfortable to wear. The shield 700 also contributes to the MRD 100 staying in place despite bruxing.

In some embodiments the shield is separate from the MRD 100, in other embodiments the shield may be connected to one of the dental plates 102, 104. In still other embodiments the shield 700 may be integrally formed with one of the dental plates 102, 104 as a one-piece unit.

FIG. 7 shows a side view of an external support for a mandibular repositioning device. The external support in the form of a head collar 800 may be used together with the MRD 100. The head collar 800 has a main adjustable support strap 802 that supports the midsection of the mandibular arch up to the frontal bone area of the head. The main supporting strap 802 may be adjustable using Velcro™, a variable hook and eye construction, or the like. The head collar 800 also includes a secondary adjustable support strap 804 that connects from the temporal area on the main strap 802 and goes around the back of the head below the crown to provide stability to the head collar 800.

The head collar 800 has, coming from the anterior, under chin section of the main supporting strap 802, an extension 806 made in a pliable rigid material on which a sleeve of comfort material will rest against the mentalis area of the lower lip and chin area. The head collar 800 holds the dental arches in their relative positions as derived by the MRD 100, and also allows voluntary opening of the mouth, yet when the muscles are relaxed, the head collar 800 holds the lower lip to the upper lip to provide passive lip seal to encourage nasal breathing.

An advantage of the mandibular repositioning devices describes herein is that they not only allow mandibular movement within natural bounds and are comfortable for the user, but also delimit the movement as appropriate for the treatment of orofacial disorders based on the configuration of the saddles and the configuration of the locating mechanism.

The MRDs described cater for lateral bruxers because the saddles are sized and shaped so that the relative mandibular positioning is maintained despite bruxing. In addition, although the substrate material for the dental plates may be made of a relatively soft (and therefore comfortable) material, the saddles may be made of a more robust material more able to withstand bruxing. Furthermore, the configuration of the locating mechanism provides play so that the device will not necessarily fail during bruxing because the location formations are less likely to become unseated from the receiving formations.

The use of a shield together with the MRD means that optimum muscular activation of the muscles in the throat is achieved when the user swallows. Because of the shape of the saddles, and in particular due to the ribs that support natural movement of the mandible relative to the maxilla, a correct vertical is supported when the user swallows, and after swallow a controlled return to the natural freeway position is enabled.

In embodiments where the saddles are removable, the MRDs are advantageously versatile as they are configurable to suit people with different bite classifications.

An advantage of the temporary type of MRD is that it provides a low-cost MRD, adjustable to suit a new user to thereby provide the user with a low-cost trial device. The user is then able to use the temporary device to ascertain its usefulness, comfort, etc., before paying for a more comprehensive and more expensive design (as shown for example in FIG. 1).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A mandibular repositioning device (MRD) which includes:
an upper dental plate shaped to fit on a person's maxillary arch;
a lower dental plate shaped to fit on the person's mandibular arch, the plates defining mating bite surfaces;
a locating mechanism for effecting antero-posterior (AP) correction of the mandibular arch relative to the maxillary arch, the locating mechanism including a locating formation extending from one of the upper dental plate and the lower dental plate and a complementary receiving formation carried by the other of the upper dental plate and the lower dental plate, the receiving formation receiving the locating formation for locating the plates relative to each other in a desired AP position of the mandibular arch relative to the maxillary arch; and
a positioning element received at each molar region of at least one of the plates to alter the bite surface, thereby effecting a natural relative movement of the dental arches
wherein the locating mechanism is positioned to lie anterior to a user's masseter muscle complex.

2. The MRD of claim 1 wherein each positioning element of at least one of the dental plates includes a bearing member shaped to effect the natural relative movement of the dental arches by guiding the movement of the person's mandible relative to the person's maxilla, the bearing member positioned so as to provide a pivot point for deflecting force resulting from masseter-temporalis contraction.

3. The MRD of claim 1 wherein the locating formation comprises a pair of spaced locating tabs extending from the one of the plates and wherein the complementary receiving formation comprises a pair of spaced receiving formations carried by the other of the plates.

4. The MRD of claim 3 wherein each locating tab is positioned at a molar region of its associated plate and each receiving formation is, similarly, positioned at a molar region of its associated plate.

5. The MRD of claim 3 wherein each receiving formation defines a slot having an AP dimension that exceeds an AP dimension of its associated locating tab to allow AP mandibular movement of the mandibular arch relative to the maxillary arch.

6. The MRD of claim 5 wherein each locating tab has a length that allows relative vertical movement of the locating tab within its respective slot without the locating tab dislodging from the slot.

7. The MRD of claim 3 wherein each of the locating tabs extends from the one of the plates from a front of the tab to a back of the tab to form a closed tab.

8. The MRD of claim 1 wherein each positioning element defines an occlusal surface, the shape of the occlusal surface being based on anatomic curvature of the occlusal alignment of teeth of the person.

9. The MRD of claim 8 wherein each positioning element of at least one of the dental plates is selected with its occlusal surface standing proud of the bite surface of its associated dental plate by a selected amount.

10. The MRD of claim 9 wherein the predetermined distance is selected based on the person's bite classification.

11. The MRD of claim 1 wherein each positioning element associated with the dental plate having the receiving formation includes an auxiliary receiving formation shaped to lie along substantially an entire length of its respective receiving formation.

12. The MRD of claim 1 further including a shield shaped to be positioned relative to a labial region of the dental plates.

13. The MRD of claim 3 wherein each dental plate comprises:
a pair of discrete molar components, each molar component carrying one of the locating formations or one of the receiving formations, and each molar component being shaped to receive one of the positioning elements; and
an adjustment mechanism that adjustably couples the pair of molar components together.

14. The MRD of claim 13 wherein the adjustment mechanism comprises a pair of coupling members, one carried by each molar component, the members being adjustably connectable together to vary the spacing of the molar components from each other.

15. A mandibular repositioning device (MRD) which includes:
an upper dental portion including a pair of discrete upper molar components, shaped to fit on a person's maxillary arch, and a first adjustment mechanism that adjustably couples the pair of upper molar components;
a lower dental portion including a pair of discrete lower molar components, shaped to fit on a person's mandibular arch, and a second adjustment mechanism that adjustably couples the pair of lower molar components together, the components of the upper and lower dental portions in register with each other defining mating bite surfaces;
a locating mechanism for effecting antero-posterior (AP) correction of the mandibular arch relative to the maxillary arch, the locating mechanism including:
a pair of locating formations, one extending from each component of one of the pair of upper molar components and the pair of lower molar components, and
a pair of complementary receiving formations, one carried by each component of the other of the pair of upper molar components and the pair of lower molar components, the receiving formations receiving the locating formations for locating the associated components of the portions relative to each other; and
a positioning element received by each molar component to alter the bite surface, thereby effecting a natural relative movement of the dental arches;
wherein the positioning element includes a bearing member positioned so as to provide a pivot point for deflecting force resulting from masseter-temporalis contraction.

16. The MRD of claim 15 wherein the bearing member is shaped to effect the natural relative movement of the dental arches by guiding the movement of the person's mandible relative to the person's maxilla.

17. A mandibular repositioning device (MRD) which includes:
an upper dental plate shaped to fit on a person's maxillary arch;
a lower dental plate shaped to fit on the person's mandibular arch, the plates defining mating bite surfaces;
a locating mechanism for effecting antero-posterior (AP) correction of the mandibular arch relative to the maxillary arch, the locating mechanism including a locating formation extending from one of the upper dental plate and lower dental plate and a complementary receiving formation carried by the other of the upper dental plate and the lower dental plate, the receiving formation receiving the locating formation for locating the plates relative to each other;
a positioning element received at each molar region of at least one of the plates to alter the bite surface, thereby effecting a natural relative movement of the dental arches; and
a shield shaped to be positioned relative to a labial region of the dental plates
wherein the positioning element includes a bearing member positioned so as to provide a pivot point for deflecting force resulting from masseter-temporalis contraction.

18. The MRD of claim 17 wherein the shield is unattached.

19. The MRD of claim 17 wherein the locating mechanism and the positioning elements are configured to allow mandibular movement.

20. The MRD of claim 17 wherein one or more of the positioning elements are removable.

21. The MRD of claim 17 wherein each positioning element associated with the dental plate having the locating formation includes an auxiliary locating formation.

22. (canceled)

23. (canceled)

24. The MRD of claim 15 wherein the locating mechanism is positioned to lie anterior to a user's masseter muscle complex.
